(19) United States
(12) Patent Application Publication
(10) Pub. No.: US 2001/0031991 A1
(43) Pub. Date: Oct. 18, 2001

(54) Circuit for producing an arbitrary defibrillation waveform

(76) Inventor: Joseph Russial, Pittsburgh, PA (US)

Correspondence Address: Michael G. Panian, Buchanan Ingersoll, P.C., One Oxford Centre, 301 Grant Street, 20th Floor, Pittsburgh, PA, 15219, US

(21) Appl. No.: 09/736,597

(22) Filed: Dec. 13, 2000

Related U.S. Application Data
(60) Provisional application No. 60/170,650, filed on Dec. 14, 1999.

(51) Int. Cl. A61N 1/39

(52) U.S. Cl. 607/5

(57) ABSTRACT

A circuit for producing an arbitrary defibrillator waveform using switching techniques which reduce the usual high current or high voltage stress on the switching element. This allows existing semiconductor devices to be used in an application previously closed to them. The result is a defibrillator able to produce desirable rectangular waveforms without the waste of energy found in existing approaches. This allows the use of a smaller energy storage capacitor for a given delivered energy. The application discussed here is a cardiac defibrillator but the techniques presented could be applied to other power conversion situations.

### RELATED APPLICATIONS

This application is related to provisional patent application Ser. No. 60/170,650, filed on Dec. 14, 1999.

### BACKGROUND OF THE INVENTION

1. Field of the Invention

This invention relates generally to the delivery of electrotherapy for the correction of cardiac arrhythmias. More particularly, this invention enables the delivery of more energy effective electrotheraputic pulses by a pulse generator having a reduced size and weight.

2. Background of the Invention

The human heart has a natural ability to beat at an appropriate contraction rate which typically varies from about 50 to 150 beats per minute. If an abnormal condition known as an arrhythmia occurs the heart's contraction rate may be excessively fast, i.e., a ventricular tachyarrhythmia such as ventricular tachycardia (VT) or ventricular fibrillation (VF). Electrotherapy is known to be capable of correcting tachyarrhythmias.

With VT or VF, it is necessary to treat the heart with high energy pulses in the range of 30 to 360 joules in order to convert the tachyarrhythmia to a normal heartrate. Arrhythmia correcting devices are known as cardioverters or defibrillators.

Since arrhythmias typically occur unexpectedly and are frequently life threatening, defibrillators that are either worn or implanted have the special advantage of being able to provide prompt and therefore effective treatment. It is desirable that devices worn by the patient be as small and lightweight as practicable.

Technology is available for correcting excessively slow heart rates (bradycardia) using implantable devices, commonly referred to as pacemakers, which deliver microjoule electrical pulses to a slowly beating heart in order to speed the heart rate up to an acceptable level. Also, it is well known to deliver high energy shocks (e.g., 180 to 360 joules) via external paddles applied to the chest wall in order to correct excessively fast heart rates, and prevent the possible fatal outcome of ventricular fibrillation or certain ventricular tachycardias. Bradycardia, ventricular fibrillation, and ventricular tachycardia are all electrical malfunctions (arrhythmias) of the heart. Each may lead to death within minutes unless corrected by the appropriate electrical stimulation.

One of the most deadly forms of heart arrhythmias is ventricular fibrillation, which occurs when the normal, regular electrical impulses are replaced by irregular and rapid impulses, causing the heart muscle to stop normal contractions and to begin to quiver. Normal blood flow ceases, and organ damage or death may result in minutes if normal heart contractions are not restored. Although frequently not noticeable to the victim, ventricular fibrillation is often preceded by ventricular tachycardia, which is a regular but fast rhythm of the heart. Because the victim has no noticeable warning of the impending fibrillation, death often occurs before the necessary medical assistance can arrive.

Because time delays in applying the corrective electrical treatment may result in death, implantable pacemakers and defibrillators have significantly improved the ability to treat these otherwise life threatening conditions. Being implanted within the patient, the device continuously monitors the patient's heart for treatable arrhythmias and when such is detected, the device applies corrective electrical pulses directly to the heart.

It is known to provide an energy delivery apparatus which includes a defibrillator electrically coupled to a patient. Such a defibrillator can produce preshaped electrical pulses such as defibrillation pulses and cardioversion pulses. The apparatus may also include an energy delivery controller electrically coupled to the patient and the converter and the defibrillator. The controller causes a converter to provide the electrical energy to the defibrillator at a specific charging rate in response to an energy level in the reservoir.

The controller causes the defibrillator to apply a selectable portion of the electrical energy in the form of electrical pulses to the body of the patient in response to the detection of a treatable condition. The preshaped electrical pulses can be approximately exponentially-shaped pulses and may be monophasic or biphasic exponential pulses. The controller measures the voltage and current being delivered to the patient during the pulse delivery period to measure the actual amount of energy being delivered to the patient.

For defibrillators, the maximum amount of stored energy is a major determinant of device size and weight. The maximum energy determines the size of the main energy storage capacitor and to a lesser extent the size of the batteries and the battery to capacitor energy converting circuitry. Additionally, the particular waveform of the defibrillation pulse has been shown to have a substantial effect on the amount of energy needed to convert VT or VF. Therefore, highly efficient defibrillation waveforms are very important in minimizing defibrillator size.

Historically, various defibrillation waveforms have been used not necessarily because they were known to be effective but because they were easy to generate with available circuitry. The earliest defibrillators were powered by alternating current (AC) and used readily available commercial power. As knowledge on the art of defibrillators increased, direct current (DC) defibrillators were shown to be more effective and various damped sine waveforms (DSW) became the accepted standard for external defibrillator waveforms. Implantable defibrillators were introduced clinically in 1980 and used truncated exponential waveforms (TEW) because of the requirement to use a relatively large inductor to generate DSWs. More recently biphasic truncated exponential waveforms (BTEW) have been found to be more energy efficient than either TEWs or DSWs and consequently BTEWs are now the standard waveforms for implantable defibrillators. Biphasic exponential pulses have a positive-going pulse segment and a negative-going pulse segment and a selected amount of electrical energy is applied to the patient during the positive-going segment and the remaining amount of the electrical energy is applied to the patient during the negative-going pulse segment. Such an apparatus is described in copending application Ser. No. 09/056,315, filed on Apr. 7, 1998, which application is assigned to the present assignee and is hereby incorporated by reference herein.

Kroll et al. and Lopin et al. in U.S. Pat. Nos. 5,391,186 and 5,733,310, respectively, both describe a modified BTEW in which the first phase has a flattened top, or, more specifically, a relatively constant first phase voltage and current. Although there are no published data comparing the efficacy of BTEWs with flattop modified BTEWs, the modified version is probably more efficient because it avoids the higher peak currents found in traditional BTEWs. Further, it is known that providing an electric current that exceeds a given current threshold value for a specified period of time defibrillates the heart. It is therefore logical to assume that a flattop BTEW will provide more energy efficient defibrillation than a conventional BTEW because of its constant current feature. Alternatively, there may be other current waveforms that will be proven to be advantageous.

It is known that the impedance in patient defibrillation circuits is highly variable with external defibrillation impedance ranging from 25 to over 100 ohms. To date, no constant current defibrillators have been commercially introduced partly because of the difficulty of producing a multi-kilowatt constant current in a small device and partly because the advantages of constant current have not been fully appreciated. The Lopin et al. patent describes a means to achieve both impedance compensation and near constant current. However, a shortcoming of the technique taught in the Lopin et al. patent is that substantial energy is wasted which is turn results in a larger than necessary defibrillator. This larger size can be quite significant if the application is for an implantable or wearable defibrillator. Accordingly, there is a need for a method that creates any desired defibrillation waveform regardless of patient impedance and that can do so without wasting energy.

Currently, the rectangular biphasic waveform is believed to be the optimum electrotherapy pulse waveform, requiring the lowest energy to defibrillate and having the lowest peak current (see FIG. 1). See U.S. Pat. No. 5,733,310, issued on Mar. 31, 1998 to Lopin et al. This patent discloses techniques used to generate a rectangular biphasic waveform in which current delivered to the patient is controlled by using a resistor connected in series between the voltage source and the patient in order to provide the desired electrotherpay to a patient having an unknown impedance. However, this wastes energy as heat in the resistor and therefore requires a larger than necessary storage capacitor to produce a given delivered energy level to the patient.

Other known techniques propose the use of output interruption techniques to increase energy delivery efficiency. These use living tissue, such as the patient's skin, as an averaging or filter element since human skin has a natural impedance or resistance. The actual waveform applied to the patient's body is not continuous but interrupted by an output switch.

Standard switching power supply techniques could be used in external defibrillators to produce a continuous output waveform of any desired shape. However, the switch element would have to withstand high voltages and/or high current. It must also support fast switching rates to reduce the magnetics to a reasonable volume and weight. Currently available switching devices generally do not meet these requirements.

It is therefore an object of the present invention to provide an electronic circuit topology which permits switching techniques to be used with currently available switch components to generate an arbitrarily shaped electrotherapy pulse waveform. The present invention does not waste energy in a resistor used to create a rectangular waveform, thus the storage capacitors which provide the voltage source for the energy in the pulse need be no larger than necessary to store the energy to be delivered. Therefore, the components can be sized to provide a device which can either be implanted or comfortably be carried on the body of a patient, such as in a wearable vest or the like, or used in a standard external defibrillation device.

### SUMMARY OF THE INVENTION

A defibrillation pulse is typically applied to a patient through switch elements which connect a first patient terminal or electrode to a voltage source and a second patient terminal or electrode to a return or ground. The present invention, however, as shown in FIG. 2 connects the patient between two voltage sources. The first is the main energy storage capacitor, while the second is a lower voltage, lower power controlled voltage source. Current through the patient is determined by the difference between the two voltage sources (V1−V2) and the patient impedance or resistance.

The voltage on the second source is preferably controlled and continuously adjusted using switching techniques to maintain the desired patient current and thus the optimal electrotherapy pulse energy to the patient's heart. Any arbitrary patient waveform can be created by appropriately adjusting the second voltage source as the first voltage source is discharged. Additionally, the energy absorbed by the second voltage source can be recovered and “pumped back” into the main storage capacitor, thus resulting in a smaller main storage capacitor bank for a given delivered energy. Since the second voltage source operates at lower voltage and power levels than the main source it is realizable with conventional switch components.

### BRIEF DESCRIPTION OF THE DRAWINGS

FIG. 1 is an illustration of a prior art defibrillator device producing an exponential waveform.

FIG. 2 is a schematic diagram of one embodiment of an energy delivery apparatus having an arbitrary waveform according to the invention herein.

FIG. 3 is a schematic diagram of an embodiment of the present invention showing one example of a control circuit for the second voltage source shown in FIG. 2.

FIG. 4 is a schematic diagram of an alternate embodiment of the present invention.

FIG. 5 shows an alternate embodiment of the present invention using a coupled flyback inductor in the switch circuitry.

FIG. 6 is a schematic diagram of a further embodiment of the present invention utilizing an integral H-bridge switch circuit to provide a biphasic electrotherapy pulse.

FIG. 7 is a still further alternate embodiment of the present invention utilizing an external H-bridge to provide a biphasic electrotherapy waveform.

FIG. 8 is a schematic diagram of an integral charger utilized in the defibrillator waveform of the present invention.

FIG. 9 shows one example of a patient electrotherapy pulse that may be produced by the present invention.

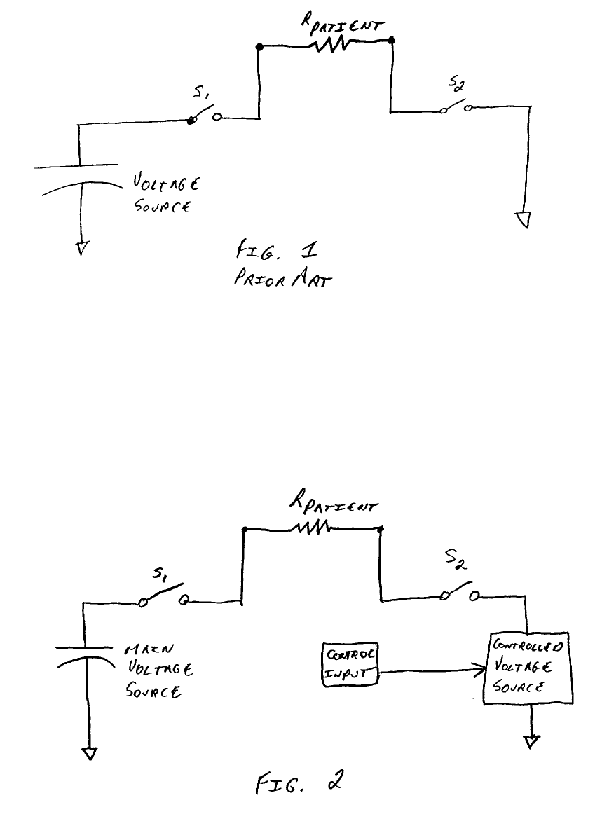

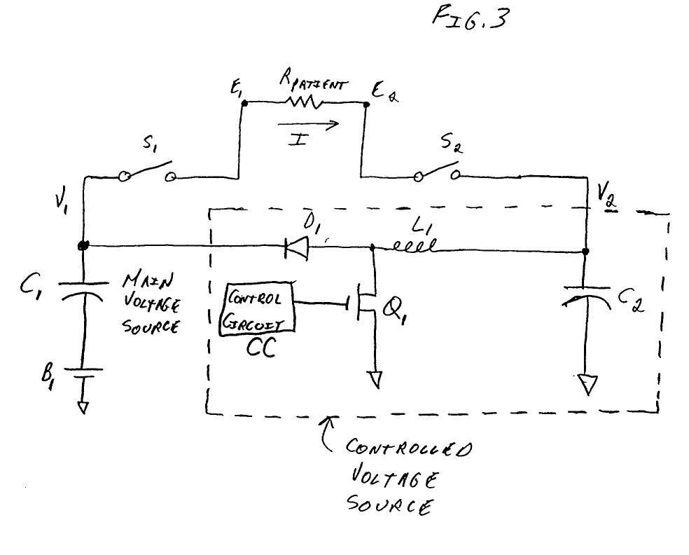

### DETAILED DESCRIPTION OF THE PREFERRED EMBODIMENTS

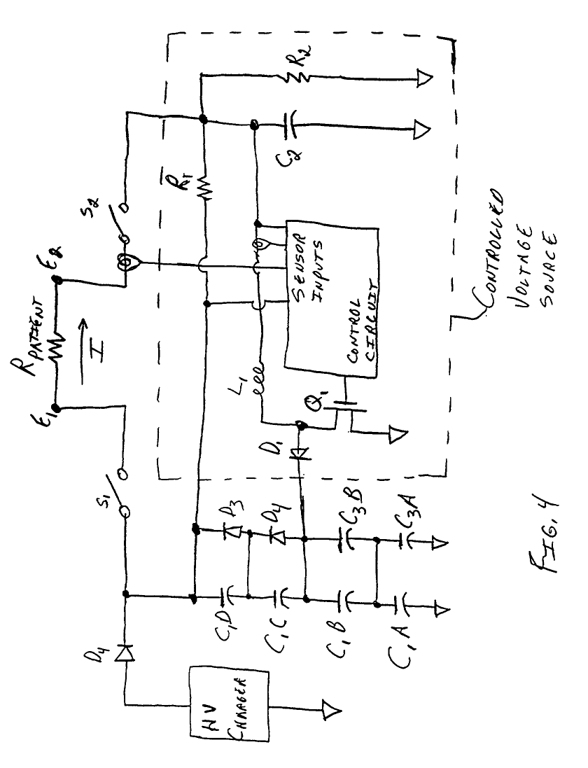

Referring now to the drawings, FIG. 3 shows a simplified schematic diagram of a patient defibrillation device of the present invention. Capacitor C1 is the main energy storage element or voltage source. It is to be understood that capacitor C1 may be comprised of a plurality of individual capacitors so connected so as to provide the desired output voltage (see, e.g., FIG. 4). A voltage source, such as battery B1, can provide the charging current for the capacitors. Switches S1 and S2 connect the patient to the defibrillator circuitry such as by patient electrodes E1 and E2. The secondary, switch controlled, voltage source is comprised of capacitor C2, inductor L1, semiconductor switch Q1, diode D1, and the control circuitry CC.

In the present invention capacitor C1 is charged to a voltage V1 and capacitor C2 is charged to a lower voltage V2. Switches S1 and S2 are closed which thereby connects the patient between the two voltages by means of the electrodes E1 and E2. Current thus begins to flow through the patient, particularly to the patient's heart. The current level I is determined by Ohm's law as (V1−V2)/Rₚₐₜᵢₑₙₜ. This current thus flows to and charges C2 causing its voltage to increase. The boost configured switching circuitry comprised of L1, Q1, D1 and the control circuit extract energy from C2 and “pump” or deliver it back into C1. As C1 discharges, the control circuit also adjusts the switching action to maintain the voltage difference V1−V2 substantially constant and thus the current or electrotherapy pulse delivered to the patient is generally maintained at the desired level.

FIG. 4 is a diagram of another preferred embodiment of the invention. As before capacitor C1 is the main energy storage element now comprised of four aluminum electrolytic capacitors C1_{A}, C1_{B}, C1_{C}, C1_{D} connected in series. Typical preferred values would be about 800 uF for each capacitor and would each be charged to approximately 400 volts for a total voltage of 1600. Switches S1 and S2 connect the patient to the defibrillator circuitry through electrodes E1 and E2. For purposes of illustration only, it will be assumed that the patient impedance is 75 ohms. Also, as before, the secondary switched voltage source is comprised of capacitor C2, inductor L1, semiconductor switch Q1, diode D1 and the control circuitry, as well as resistors R1 and R2.

The main storage capacitor C1 is charged to 1600 volts by a high voltage charger HV through isolating diode D4. Capacitor C2 is preferably a small high frequency aluminum electrolytic capacitor with a value of about preferably 10 uF. Resistors R1 and R2 form a voltage divider to charge C2 via high voltage charger HV to an initial voltage of about 400 volts. Closing switches S1 and S2 connects the patient between the two voltages and provides the electrotherapy pulse to the patient through electrodes E1 and E2. The voltage across the patient is 1600−400=1200. Current thus begins to flow through the patient, and particularly to the patient's heart, to provide the defibrillator electrotherapy pulse. The patient current level is therefore determined by Ohm's law to be (1600−400)/75=16 amps. After passing through the patient, this current then passes on to and charges C2, causing its voltage to increase. The boost configured switching circuitry comprised of L1, Q1, D1, and the control circuit extract energy from C2 and deliver it back into charging capacitors C1ₐ and C1_{b}. Capacitors C3ₐ and C3_{b} are preferably small value low impedance ceramic capacitors to absorb the high frequency current pulses from the inductor's discharge. As C1 discharges, it delivers less voltage to the patient. The control circuit automatically adjusts the switching action to maintain the voltage difference between C1 and C2 at 1200 volts, and thus the patient electrotherapy pulse current is maintained at the desired level of 16 amps. Since C1ₐ and C1_{b} have their charge partially replaced by the switching action, their voltage decays slower than C1_{c} and C1_{d}. At some point C1_{c} and C1_{d} have no charge remaining and diodes D2 and D3 prevent them from becoming reverse biased.

An advantage of the present invention is that it can automatically and substantially constantly deliver the desired electrotherapy pulse of 16 amps to a patient of unknown impedance. For example, the initial voltage on C2 can be set for the lowest expected patient impedance such as 25 ohms (i.e., C2 is 1200 volts, since (C1600−1200/25)=16). Once the pulse is initiated the voltage delivered by C2 can be ramped down quickly by the control circuitry which automatically detects the resistance of the patient via the sensor inputs until the patient current reaches the desired level.

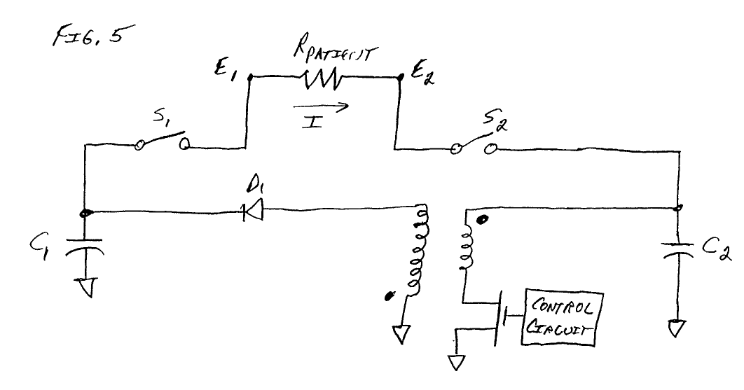

In addition to the embodiments discussed above, the present invention can incorporate other switch circuitry components to provide added advantages. For example, a transformer, in the form of a coupled flyback inductor, can be used in place of the inductor. Such a coupled flyback inductor is shown in FIG. 5. This component will also operate to transfer the voltage from the second voltage source to the first voltage source as the electrotherapy pulse is being delivered through the patient.

The present invention can also be configured to provide a biphasic waveform having a positive going segment and a negative going segment in a second pulse. An integral H-bridge circuit as shown in FIG. 6 can provide this function. In this embodiment diode D1 is replaced by an insulated gate bipolar transistor (IGBT) or MOSFET Q2 with either an internal or external anti-parallel diode D5 and a further semiconductor switch S3. During the forward (positive) first phase switch S3 is open, while during the reverse (negative) second phase switch S1 is opened and switch S3 is closed and IGBT/MOSFET Q2 is turned on.

As a further refinement to the present invention, a negative voltage may be induced on the second voltage source C2 in order to completely drain the voltage from the main voltage source C1. If the regulator components are reconfigured to allow the voltage on second voltage source C2 to go negative, the current through the patient can be controlled until the entire charge is drawn from main voltage source C1 instead of dropping out of regulation when the voltage on C2 reaches zero. This makes the defibrillator additionally energy efficient as a typical TEW defibrillator still has approximately 12% of the stored energy remaining at the end of the pulse. Alternatively, this technique could also be used to produce a regulated second phase for a biphasic pulse wherein switch S1 is opened and switch S3 is closed (see FIG. 6).

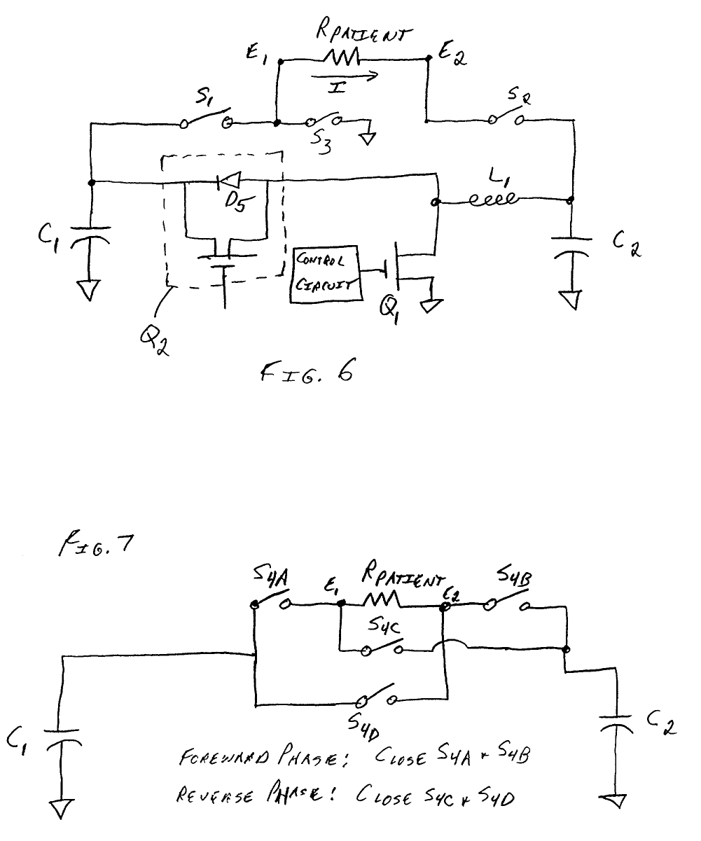

Alternatively, generation of the second (negative) phase pulse can be accomplished by using an external H-bridge. This uses the basic circuit as shown in FIG. 3 but switches S1 and S2 are replaced with an H-bridge comprises switches S4_{A}, S4_{B}, S4_{C} and S4_{D} as shown in FIG. 7. This allows biphasic pulse generation and regulation of the second (reverse) phase if desired. During the positive pulse, switches S4_{A} and S4_{B} are closed, while during the negative pulse, switches S4_{C} and S4_{D} are closed.

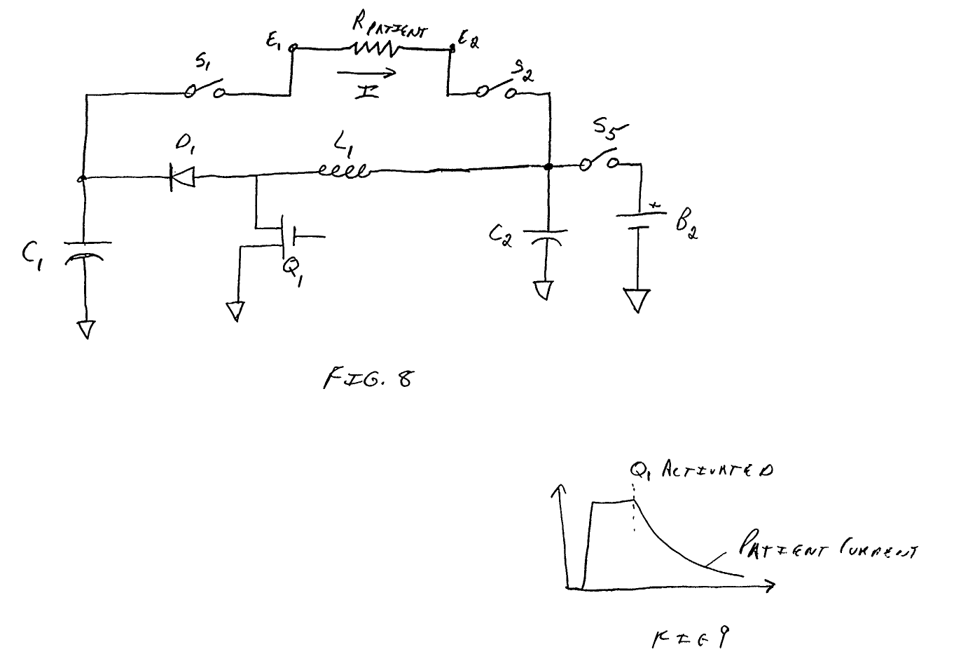

In the embodiment shown in FIG. 8, an integral charger can be used which uses a single inductor L1 as both the charging inductor and the regulating inductor. Generally, magnetic components are relatively physically large and may in turn result in a larger than desired defibrillator device. This circuit configuration eliminates the need for a separate charging inductor or transformer, thereby eliminating components so as to reduce the overall size and weight of a defibrillator. While switch S5 has been added, they are still small relative to the eliminated components.

In another embodiment of the present invention, the individual capacitors comprising the main voltage source can be connected in parallel rather than in series in order to optimize the circuit for various patient impedances. If the impedance of the patient is known or can be estimated before the application of electrotherapy pulse, the individual elements of C1 can be arranged to optimize the pulse delivery. Series arrangements of the individual capacitors would be more desirable for higher patient impedances, while a parallel arrangement would be preferred for lower impedances.

In a further extension of the present invention, an exponential conclusion to the first phase can be created. If at some point during the discharge cycle the switching action to semiconductor switch Q1 is disabled and it is turned on continuously, the electrotherapy pulse will go to a conventional exponential discharge. This configuration could be utilized to generate a waveform as shown in FIG. 9 that eliminates the high peak currents usually associated with exponential waveforms.

A falling current waveform could be produced by reducing the charge removal from main voltage source C1 for a time and then permitting this voltage to increase. If the switching action to Q1 is disabled for a period of time, the voltage on second voltage source C2 will increase as charge flows into it. This reduces the voltage difference (V1−V2) across the patient and therefore reduces the patient current. This technique can be used to produce waveforms where the current falls in a predetermined manner.

A unique advantage of the present invention is that, within certain limits, any arbitrary waveform can be generated to provide defibrillation to a patient. In this embodiment, it is not a requirement that the difference value V1−V2 remain constant. Therefore, the present invention can be easily re-configured to produce any waveform determined by medical science to be most advantageous for a patient, in terms of delivering the desired energy level while minimizing adverse affects to the patient and/or the patient's heart.

The invention disclosed herein enables the use of high voltage capacitors having relatively low capacitance. For example, it is estimated that 2000 volt 85 or 15 mfd thin film (polyvinilidine fluoride) capacitor could provide delivered energy of 150 and 25 joules respectively for an external or implantable defibrillator. The waveform could have a desirable duration of 10 to 12 milliseconds instead of the less desirable 2 or 3 millisecond duration that would occur with a typical RC time constant. Utilizing a flattop modified BTEW, these energies are expected to be highly effective and the energy density for this type of capacitor is expected to be as high 5.5 joules per cubic centimeter which enables size reduction due both to improved energy effectiveness and to improved capacitor energy density.

While specific embodiments of practicing the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting to the scope of the invention which is to be given the full breadth of the following claims, and any and all embodiments thereof.

1. An electrotherapy circuit for producing a defibrillation waveform to a patient having an impedance, the circuit comprising:
a main voltage source;
a first patient electrode operatively connected between the patient and the first voltage source;
a second voltage source;
a second patient electrode operatively connected between the patient and the second voltage source;
a control circuit operatively connected between said second voltage source and said first voltage source such that an electrical circuit is created from the first voltage source to the second voltage source via the patient; and
means for triggering an electrotherapy pulse to the patient.

2. The electrotherapy circuit of claim 1, further comprising a boost network including an inductor and a diode each connected in series between said second voltage source and said first voltage source and a transistor operatively connected with a controller for discharging the second voltage source into the first voltage source.

3. The electrotherapy circuit of claim 1, wherein the control circuit comprises a boost switch circuit.

4. The electrotherapy circuit of claim 3, wherein the first voltage source has a voltage V1 and the second voltage source has a voltage V2, less than V1, wherein the controller maintains a difference between V1 and V2 substantially constant.

5. The electrotherapy circuit of claim 1, wherein the circuit produces an arbitrary waveform.

6. A method of providing an electrotherapy pulse to a patient comprising:
connecting a first electrode to the patient;
providing a first voltage source for transmitting a first voltage to the first electrode;
connecting a second electrode to the patient;
connecting a second voltage source having a second voltage to the second electrode;
connecting a controller to the second voltage source; and
initiating an electrotherapy pulse to the patient by discharging a first voltage source through the first patient electrode such that the difference between the first voltage source and the second voltage source is maintained substantially constant.

7. An electrotherapy circuit and method as described and shown herein.

8. An electrotherapy circuit as shown in FIG. 3.

9. An electrotherapy circuit as shown in FIG. 4.

10. An electrotherapy circuit as shown in FIG. 5.

11. An electrotherapy circuit as shown in FIG. 6, including an internal H-bridge circuit.

12. An electrotherapy circuit as shown in FIG. 7, including an external H-bridge circuit.

13. An electrotherapy circuit as shown in FIG. 8 and comprising a single inductor for both charging the circuit and delivering an electrotherapy pulse to a patient.
